(19) United States
(12) Patent Application Publication
(10) Pub. No.: US 2009/0325235 A1
(43) Pub. Date: Dec. 31, 2009

(54) THERMOACTIVE SIVagm SAB REVERSE TRANSCRIPTASE

(75) Inventor: Baek KIM, Rochester, NY (US)

Correspondence Address: BLANK ROME LLP, WATERGATE, 600 NEW HAMPSHIRE AVENUE, N.W., WASHINGTON, DC, 20037, US

(73) Assignee: University of Rochester Medical Center, Rochester, NY (US)

(21) Appl. No.: 12/145,734

(22) Filed: Jun. 25, 2008

(51) Int. Cl. C12P 19/34

(52) U.S. Cl. 435/91.2; 435/91.51

(57) ABSTRACT

Methods and kits performing reverse transcription and RT-PCR reactions having high fidelity, processivity and DNA polymerase activity are described. The methods involve performance of reverse transcription at an increased temperature with a reverse transcriptase from Simian Immunodeficiency Virus-agm.sab or a variation thereof. The kits of the present invention include a reverse transcriptase from Simian Immunodeficiency Virus-agm.sab or a variation thereof, a DNA polymerase capable of amplifying cDNA under conditions suitable for polymerase chain reaction, and the reagents necessary to carry out both processes.

### STATEMENT OF GOVERNMENT INTEREST

The subject matter of this application was made with support from the United States Government under Grant No. R01 AI049781-01A1 from the National Institutes of Health. The United States Government has certain rights in the invention.

### FIELD OF THE INVENTION

The subject matter of the present invention relates to methods for the synthesis of DNA from RNA templates.

### BACKGROUND OF THE INVENTION

Molecular analysis of RNA molecules relies heavily on reverse transcription, which preserves molecular information of chemically unstable RNAs as stable DNAs that can be used in various analytical methodologies. The synthesized DNA molecules are often further amplified by polymerase chain reaction (PCR), which greatly enhances the efficiency of RNA analysis (7; 9). Reverse-transcriptase PCR (RT-PCR) has been widely used for various RNA analyses such as diagnostic detection (17-19, 23), genotyping of various viral RNAs in infected samples (5; 28) quantitation of gene expression (QRT-PCR) (1, 6, 7, 24, 25), and construction of cDNA libraries from various biological organisms (20).

Reverse transcription of RNA is catalyzed by retroviral DNA polymerases called reverse transcriptases (22). Unlike other DNA polymerases, reverse transcriptases synthesize DNA from both DNA and RNA templates. All retroviral reverse transcriptases, such as murine leukemia virus (MuLV) and human immunodeficiency virus type 1 (HIV-1), lack a 3′→5′ proofreading exonuclease, which contributes to the accuracy of other DNA polymerases (26). For this reason, reverse transcription is highly error prone. Mutations created during the reverse transcription step of the RT-PCR are delivered to the final products to be analyzed; this interferes with the correct analysis of RNA molecules.

Another deleterious aspect of reverse transcription is its use of RNA as a template. RNA can form various types of secondary structures that interfere with processive DNA synthesis (4, 15, 16). RNA molecules containing secondary structures, called pause sites, are difficult to extend to full-length DNA products. Indeed, accuracy and processivity of reverse transcription are key elements for the improvement of RNA analyses.

It is well known that, when reverse transcriptase pauses during cDNA synthesis, it can jump to other templates and continue synthesis, resulting in the recombination of the sequences encoded in two or more different templates. These homologous recombination events often generate unwanted artifact sequences combinations between multiple templates in a single reaction.

Previously, the inventor has reported that an increase in temperature greatly enhances both the accuracy and the processivity of reverse transcription, with minimum impact on the DNA polymerase activity of reverse transcriptases (29). The increase in processivity allows for the production of longer cDNAs from the reverse transcription reaction. However, typical reverse transcriptase enzymes have poor DNA polymerase activity at the higher temperatures necessary to achieve these enhancements in accuracy and processivity. As such, there remains a need in the art for reverse transcriptases that are capable of providing higher DNA polymerase activity at the elevated temperatures that enhance accuracy and processivity.

### SUMMARY OF THE INVENTION

It is an object of the present invention to provide a process for producing cDNA from RNA through use of a reverse transcriptase from Simian Immunodeficiency Virus-agm.sab or a variation thereof. The use of these reverse transcriptases allows for high DNA polymerase activity to be maintained at temperatures where reverse transcriptase fidelity and processivity are increased. The processes of the present invention allow for more efficient methods to produce long and accurate cDNAs.

It is a further object of the present invention to provide a process for producing cDNA from RNA using the reverse transcriptases described above in an reverse transcriptase—polymerase chain reaction (RT-PCR) procedure. Efficient and accurate reverse transcription can be performed followed by amplification of the cDNAs produced.

It is a still further object of the present invention to provide a kit for the performance of RT-PCR which contains a reverse transcriptase from Simian Immunodeficiency Virus-agm.sab or a variation thereof, a DNA polymerase capable of amplifying cDNA under polymerase chain reaction conditions and reagents suitable for the performance of both reverse transcription and polymerase chain reaction.

### DESCRIPTION OF THE DRAWINGS

FIG. 1(A) is a schematic of the primer and template used in the reverse transcription reaction described in Example 2.

FIG. 1(B) is polyacrylamide gel showing the reverse transcription primer extension described in Example 2.

FIG. 2 is a plot of the percent of primer fully extended vs. time as taken from the polyacrylamide gel shown in FIG. 3.

FIG. 3 is a polyacrylamide gel showing the thermal effect of reverse transcriptase fidelity as described in Example 3.

FIG. 4 is a polyacrylamide gel showing the thermal effect of reverse transcriptase processivity as described in Example 4.

### DETAILED DESCRIPTION OF THE INVENTION

The present invention provides improved methods and kits for the conversion of isolated RNA into DNA. The methods and kits of the present invention may be used in performing reverse-transcriptase polymerase chain reactions (RT-PCRs) for the synthesis of cDNA from RNA.

The method of the present invention allow for the efficient performance of RT-PCR at reaction temperatures higher than the temperature typically used for RT-PCR procedures. Performing RT-PCR at higher temperatures than the typically used temperature of 37° C. has been shown to significantly increase both the accuracy and the processivity of reverse transcription (29).

The present invention provides a method for performing RT-PCR and reverse transcription using a reverse transcriptase from Simian immunodeficiency virus African green monkey sabaeus (SIV-agm.sab) and variants thereof. Reverse transcriptase enzymes of this type allow for higher DNA polymerase activity at temperatures that allow for increased accuracy and processivity of reverse transcription.

In one embodiment, the methods of the present invention are modified versions of standard RT-PCR protocols (see, for example http://www.neb.com/nebecomm/products/productE6400.asp) Typically, the standard RT-PCR protocols are modified so that the reverse transcription step of the protocol is performed at a temperature of about 45°-65° C. In certain embodiments of the present invention, the reverse transcription step of the RT-PCR protocol is performed at 55°-60° C. The following steps for amplifying the reverse transcribed cDNA can be performed as is well known in the art. Other steps in the RT-PCR protocol may be modified or changed in order to provide for efficient production of the specific cDNA sequences desired as is well known in the art.

In general, in the methods of the present invention, the reverse transcription step or steps can be performed at a temperature of between about 45° C. and about 65° C. In certain embodiments of the present invention, reverse transcription is performed at a temperature of between about 55° C. and about 60° C. Typically, the reverse transcriptase step of the present invention is incubated at this temperature for between about 1 and about 20 minutes. cDNA produced from reverse transcription may be isolated using methods well known in the art, such as gel electrophoresis and column chromatography.

One example of a RT-PCR protocol that can be used with the methods of the present invention is the increased temperature protocol provided by Malboeuf, et al. (29), which is hereby incorporated herein. The protocol provided by Malboeuf may be followed substantially as it is described, with a reverse transcriptase from SIV-agm.sab, or a variant thereof, used for the reverse transcription step.

In other embodiments of the invention, the methods of the present invention are non-PCR reverse transcription methods. It should be apparent of one of skill in the art that the reverse transcription methods described herein can be substituted for any reverse transcription process known in the art, whether that reverse transcription step stands alone or is part of a more elaborate process.

The methods of the present invention provide for methods of performing reverse transcription using reverse transcriptase from SIV-agm.sab, or variants thereof. In certain embodiments of the invention, the SIV-agm.sab reverse transcriptase is a protein having the amino acid sequence of SEQ ID NO: 2. In other embodiments, the SIV-agm.sab may be a variant of SEQ ID NO: 2, such as a protein having a sequence with about 90% or greater sequence similarity to SEQ ID NO: 2. It should be apparent to one of skill in the art that any variant of SEQ ID NO: 1 allowing for higher DNA polymerase activity falls within the scope of the present invention.

The reverse transcriptase of the present invention may be provided in any manner. It may be produced using suitable recombinant methods and purified or isolated from the host virus, methods of doing both of which are well known in the art. If the reverse transcriptase is to be produced in a recombinant manner, it may be produced from a nucleic acid sequence having the substantially the same sequence as SEQ ID NO:1. Alternatively, it may be produced from sequences having greater than about 90% homology to SEQ ID NO:1.

In other embodiments of the present invention, a kit for performing RT-PCR is presented. The kits of the present invention contain a reverse transcriptase from SIV-agm.sab or a variant thereof along with a polymerase typically used in the performance of PCR amplification of DNA, such as Taq polymerase or another DNA polymerase with a temperature optimum at around 70° C. The kits of the present invention will also include the other necessary reagents needed for the performance of RT-PCR, such as the presence of deoxynucleotides and buffers that are known in the art for use with reverse transcriptase and/or PCR reactions. In certain embodiments of the kits of the present invention, the amount of deoxynucleotides and the types of buffers are suitable for the performance of the entire RT-PCR reaction in one vessel without the need for additional reagents.

The following examples are meant for illustrative purposes only, and are not intended to limit the scope of the invention as claimed below. There may be other variants not explicitly described in this application to which it would be apparent to one of skill in the art to fall within the scope of the claims below.

### EXAMPLES

### Example 1

### Reverse Transcription Reaction Protocol

A 38-mer template (5′-GCUUGGCUGCAGAAUAU UGCUAGCGG GAAUUCGGCGCG-3′, concentration 50 nM) was annealed to a 5′ P³² end labeled 23-mer primer (5′-CGCGCCGAATTCCCGCTAGCAAT-3′, concentration=20 nM), was premixed with 4× reaction buffer (100 nM Tris-HCl, pH 8.0, 400 mM KCl, 8 mM DTT, 0.4 mg/ml bovine serum albumin), and 250 mM dNTPs in 18 μL. cDNA synthesis was initiated by adding 2 μl SIVagm SAB RT (25 nM) followed by incubation for 5 min at 55˜60° C. The reaction was terminated by heating at 95° C. for 3 min.

### Example 2

### Thermal Effect on the Reverse Transcription of SIV.agm-sab Reverse Transcriptase

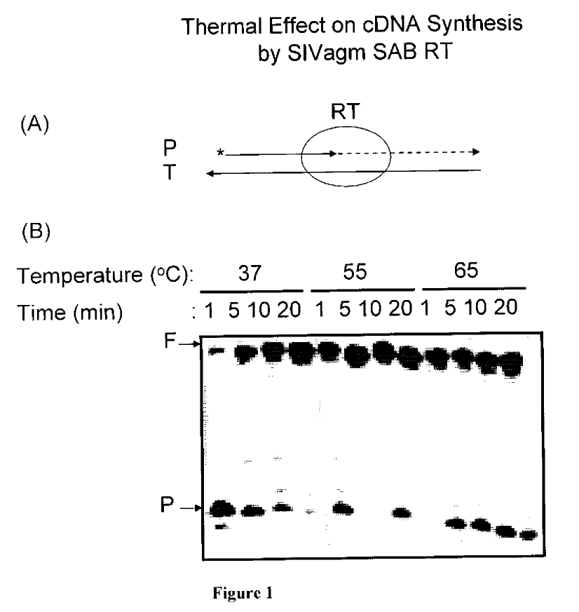

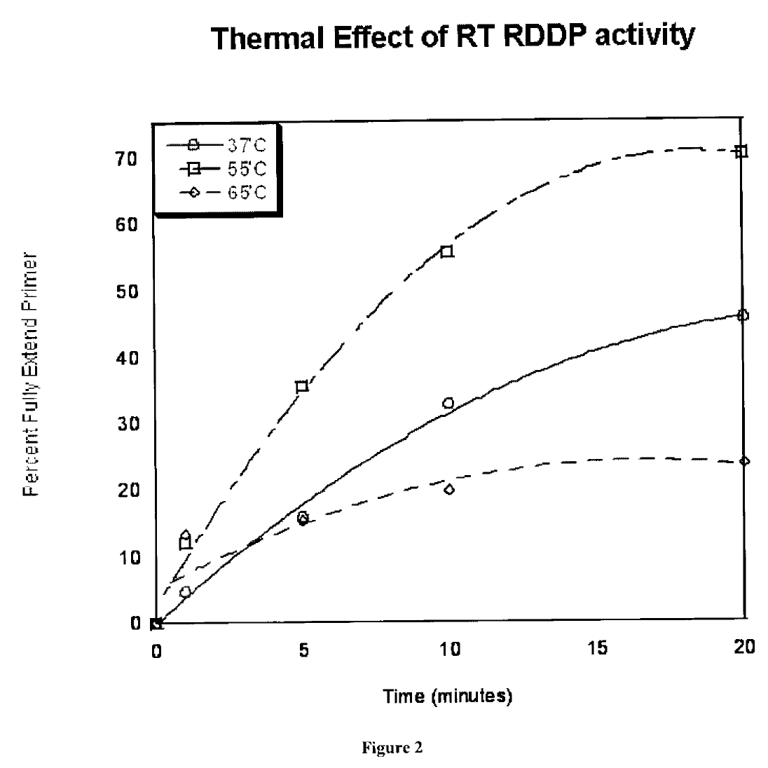

The primer extension reaction performed is illustrated schematically in FIG. 1(A). A 5′ end ³²P-labeled (*) 23-mer primer (P, 5′-CGCGCCGAATTCCCGCTAGCAAT-3′) was annealed the to a 38-mer RNA template (T, 5′-GCUUGGCUGCAGAAUAU UGCUAGCGG GAAUUCGGCGCG-3′: template: primer ratio=2.5:1) and was extended by SIVagm SAB RT. As shown in FIG. 1(B) the T/P was extended by SIVagm SAB RT with 250 mM dNTP at 37, 55 and 65° C., and the reactions were terminated at 1, 5, 10, and 20 min incubations. The reaction products were analyzed by 14% urea-denatuting gel electrophoresis. F: 38 nucleotide long fully extended product, P: 23-mer unextended primer. A plot of the percent of primer fully extended vs. time is shown in FIG. 2.

### Example 3

### Thermal Effect of Reverse Transcriptase Fidelity

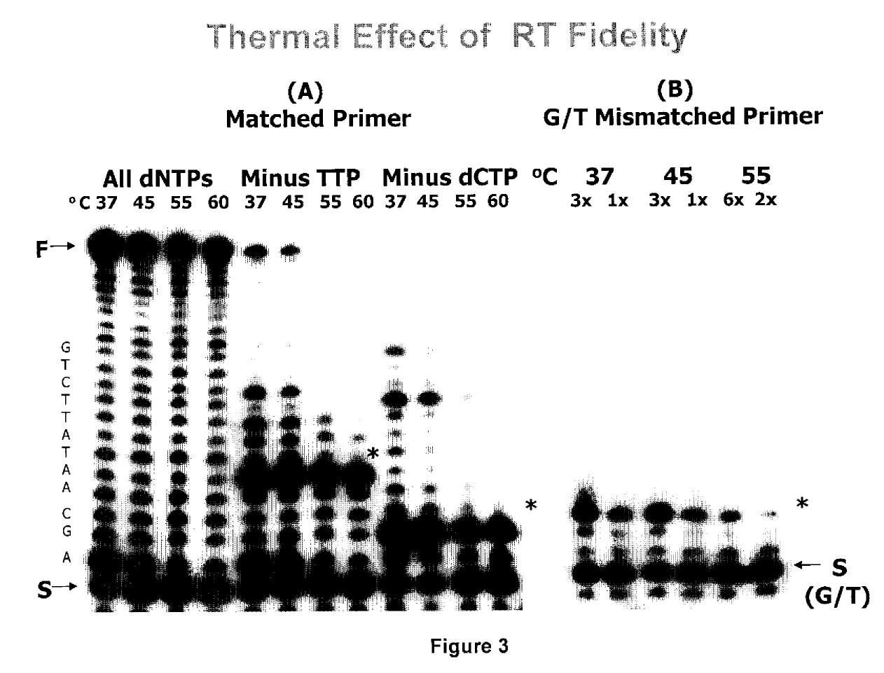

A misincorporation assay with a matched primer was performed as follows: the ³²P-labeled 17-mer matched primer (“S”) annealed to a 38-mer RNA template was extended by MuLV RT (15 nM) at 37° C., 45° C., 55° C., and 60° C. for 3 min in the presence of either all four dNTPs or only three complementary dNTPs (minus TTP and minus dCTP). As determined by amounts of the fully extended primer (“F”) in all dNTPs, reverse transcriptase activity of RT was reduced to 65% at 55° C. The sites with “*” indicate the stop sites where the deleted dNTPs would be incorporated into the reactions with only three dNTPs. In the assay with matched primer and only three dNTPs, the higher efficiency of elongation of terminated primer beyond the stop sites reflected the lower fidelity of the reverse transcriptase protein assayed, as is shown in FIG. 3(A).

An extension of mismatched primer assay was performed as follows: the 32P-labeled 16-mer G/T mismatched primer └“S (G/T)”┘ annealed to a 38-mer RNA template was extended by RT at 37° C., 45° C., and 55° C. for 3 min. In this reaction, 2-fold higher concentrations (6′ and 2′) of RT were used at 55° C. to compensate for the reduction of the reverse transcriptase activity at 55° C. The extension reactions with mismatched primer were performed in the presence of three dNTPs (minus dCTP), and the mismatched primer could be extended only up to the first stop site [“*” in FIG. 3(B)]. In the assay with mismatched primer, the higher efficiency of elongation of the mismatched primer reflected the lower fidelity of the reverse transcriptase protein, as is shown in FIG. 3(B). The reactions were analyzed by 14% denaturing gel electrophoresis. The DNA sequence of the first 12 nucleotides of the extended part of the primer is shown in FIG. 3(A).

### Example 4

### Thermal Effect of Reverse Transcriptase Processivity

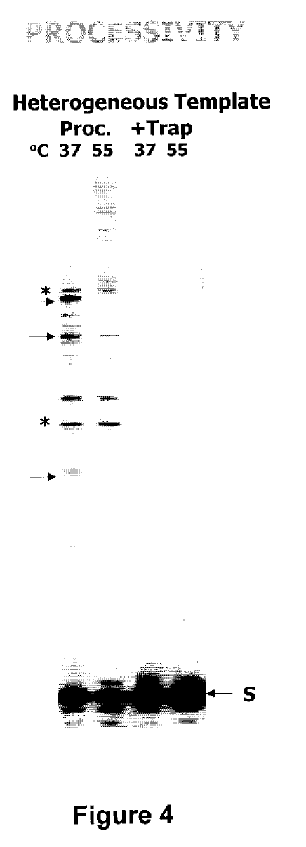

Heterogeneous RNA template encoding an HIV-1 Pol sequence, annealed to the ³²P-labeled 21-mer 3305 primer, was used in this processivity assay. Processivity (Proc): template-primer (T/P) was first preincubated with MuLV reverse transcriptase proteins at 37° C. or 55° C. for 3 min, and then the extension reactions were initiated by adding trap mixture containing dNTPs, a molar excess of poly(rA)/oligodT, and heparin at 37° C. or 55° C. for 3 min. This condition allowed only a single round of primer extension by reverse transcriptase as confirmed by two control experiments (+Trap and −Trap). +Trap: RNA T/P was first pre-mixed with the trap mixture, and the extension reactions were initiated by adding MuLV reverse transcriptase protein at 37° C. or 55° C. for 3 min. This condition blocked reverse transcriptase binding to the labeled TIP and extending the primer, supporting a single round of primer extension in the processivity reaction. In addition, RNA T/P was also pre-mixed with reverse transcriptase proteins and then the extension reactions were initiated by only dNTPs without trap (−Trap control) at 37° C. or 55° C. for 3 min. This condition allowed multiple rounds of primer extension by reverse transcriptase proteins, generating more and much longer extended products (data not shown). All reactions were analyzed by 10% denaturing gel electrophoresis. As is shown in FIG. 4, the pause sites sensitive to temperature were marked by arrows, and the pause sites insensitive to temperature were marked by “*”.

### REFERENCES

1. Bieche, I., C. Nogues, V. Paradis, M. Olivi, P. Bedossa, R. Lidereau, and M. Vidaud. 2000. Quantitation of hTERT gene expression in sporadic breast tumors with a real-time reverse transcription-polymerase chain reaction assay. Clin. Cancer Res. 6:452-459.
2. Boosalis, M. S., J. Petruska, and M. F. Goodman. 1987. DNA polymerase insertion fidelity. Gel assay for site-specific kinetics. J. Biol. Chem. 262:14689-14696.
3. Cheynier, R., S. Gratton, J. P. Vartanian, A. Meyerhans, and S. Wain-Hobson. 1997. G®A hypermutation does not result from polymerase chain reaction. AIDS Res. Hum. Retroviruses 13:985-986.
4. DeStefano, J. J., R. G. Buiser, L. M. Mallaber, P. J. Fay, and R. A. Bambara. 1992. Parameters that influence processive synthesis and site-specific termination by human immunodeficiency virus reverse transcriptase on RNA and DNA templates. Biochim. Biophys. Acta 1131:270-280.
5. Engelbrecht, S., T. L. Smith, P. Kasper, E. Faatz, M. Zeier, D. Moodley, C. G. Clay, and E. J. van Rensburg. 1999. HIV type 1 V3 domain serotyping and genotyping in gauteng, mpumalanga, kwaZulu-natal, and western cape provinces of South Africa. AIDS Res. Hum. Retroviruses 15:325-328.
6. Foley, K. P., M. W. Leonard, and J. D. Engel. 1993. Quantitation of RNA using the polymerase chain reaction. Trends Genet. 9:380-385.
7. Freeman, W. M., S. J. Walker, and K. E. Vrana. 1999. Quantitative RT-PCR: pitfalls and potential. BioTechniques 26:112-122.
8. Fry, M. and L. A. Loeb.1992. A DNA polymerase alpha pause site is a hot spot for nucleotide misinsertion. Proc. Natl. Acad. Sci. USA 89:763-767.
9. Hagen-Mann, K. and W. Mann. 1995. RTPCR and alternative methods to PCR for in vitro amplification of nucleic acids. Exp. Clin. Endocrinol. Diabetes 103:150-155.
10. Huang. M. M., N. Arnheim, and M. F. Goodman. 1992. Extension of base mispairs by Taq DNA polymerase: implications for single nucleotide discrimination in PCR. Nucleic Acids Res. 20:4567-4573.
11. Kamath-Loeb, A. S., A. Hizi, H. Kasai, and L. A. Loeb. 1997. Incorporation of the guanosine triphosphate analogs 8-oxo-dGTP and 8-NH2-dGTP by reverse transcriptases and mammalian DNA polymerases. J. Biol. Chem. 272:5892-5898.
12. Kim, B. 1997. Genetic selection in Escherichia coli for active human immunodeficiency virus reverse transcriptase mutants. Methods 12:318-324.
13. Kim, B., T. R. Hathaway, and L. A. Loeb. 1998. Fidelity of mutant HIV-1-1 reverse transcriptases: interaction with the single-stranded template influences the accuracy of DNA synthesis. Biochemistry 37:5831-5839.
14. Kim. B., J. C. Ayran, S. G. Sagar, E. T. Adman, S. M. Fuller, N. H. Tran, and J. Horrigan. 1999. New human immunodeficiency virus type 1 reverse transcriptase (HIV-1-1RT) mutants with increased fidelity of DNA synthesis: accuracy, template binding, and processivity. J. Biol. Chem. 274:27666-26673.
15. Kim, J. K., C. Palaniappan, W. Wu, P. J. Fay, and R. A. Bambara. 1997. Evidence for a unique mechanism of strand transfer from the transactivation response region of HIV-1. J. Biol. Chem. 272:16769-16777.
16. Klarmann, G. J., C. A. Schauber, and B. D. Preston. 1993. 1Template-directed pausing of DNA synthesis by HIV-1 reverse transcriptase during polymerization of HIV-1 sequences in vitro. J. Biol. Chem. 268:9793-9802.
17. McGinniss, M. J., D. H. Brown, L. W. Burke, J. T. Mascarello, and M. C. Jones.1997. Ring chromosome X in a child with manifestations of Kabuki syndrome. Am. J. Med. (Genet. 70 37-42.
18. Metcalf, T. G., J. L., Melnick, and M. K. Estes. 1995. Environmental virology: from detection of virus in sewage and water by isolation to identification by molecular biology—a trip of over 50 years. Annu. Rev. Microbiol. 49:461-487.
19. Nakamura, S., S. Katamine, T. Yamamoto, S. K. Foung, T. Kurata, Y. Hirabayashi, K. Shimada, S. Hino et al. 1993. Amplification and detection of a single molecule of human immunodeficiency virus RNA. Virus Genes 7:325-333.
20. Peterson, L. A., M. R. Brown, A. J. Carlisle, E. C. Kohn, L. A. Liotta, M. R. Emmert-Buck, and D. B. Krizman. 1998. An improved method for construction of directionally cloned cDNA libraries from microdissected cells. Cancer Res. 58:5326-5328.
21. Preston, B. D., B. J. Poiesz, and L. A. Loeb. 1958. Fidelity of HIV-1 reverse transcriptase. Science 242: 1168-1171.
22. Skalka, A. M. and S. P. Goff. 1993. Reverse Transcriptase, p. 1-4. CSH Laboratory Press, Cold Spring Harbor, N.Y.
23. Schiff, E. R., M. de Medina, and R. S. Kahn. 1999. New perspectives in the diagnosis of hepatitis C. Semin. Liver Dis. 19:3-15.
24. Sunday, M. E. 1995. Differential display RTPCR for identifying novel gene expression in the lung. Am. J. Physiol. 269:273-284.
25. Walker, K. B. 1998. Detection and analysis of cytokine mRNA in tissues and cell lines. J. Immunol. Methods 212:11-123.
26. Williams, K. J. and L. A. Loeb. 1992. Retroviral transcriptases: error frequencies and mutagenesis, p. 165-180. In J. J. Holland (Ed.), Current Topics in Microbiology and Immunology: Genetic Diversity of RNA Viruses. Spriner-Verlag, Berlin Heidelberg.
27. Wisniewski, M., C. Palaniappan, Z. Fu, S. F. Le Grice, P. Fay, and R. A. Bambara. 1999. Mutations in the primer grip region of HIV reverse transcriptase can increase replication fidelity. J. Biol. Chem. 274:28175-28184.
28. Zekri, A. R., A. A. Bahnassy, S. M. Shaarawy, O. A. Mansour, M. A. Maduar, H. M. Khaled, and O. El-Ahmadi. 2000. hepatitis C virus genotyping in relation to neu-oncoprotein overexpression and the development of hepatocellular carcinoma. J. Med. Microbiol. 49:89-95.
29. Malboeuf C M. Isaacs, S J, Tran, N H and Kim, B, 2001, Thermal Effects on Reverse Transcription: Improvement of Accuracy and Processivity in cDNA synthesis.

What is claimed is:

1. A process for producing cDNA from isolated RNA, comprising:
providing isolated RNA from which cDNA is desired to be produced;
contacting the RNA with a suitable buffer, a suitable amount of deoxynucleotides and a reverse transcriptase from Simian Immunodeficiency Virus-agm.sab to form a mixture; and
incubating the mixture at an incubation temperature of about 45° C. to about 65° C. for an incubation period of between about 1 minute to about 20 minutes; wherein the RNA is reverse transcribed to cDNA.

2. The process of claim 1, wherein the reverse transcriptase has an amino acid sequence comprising an amino acid sequence having at least about 90% sequence similarity to SEQ ID NO:2.

3. The process of claim 1, wherein the reverse transcriptase has an amino acid sequence comprising an amino acid sequence having at least about 95% sequence similarity to SEQ ID NO:2.

4. The process of claim 1, wherein the reverse transcriptase has an amino acid sequence comprising SEQ ID NO.2

5. The process of claim 1, wherein the incubation temperature is about 55° C. to about 60° C.

6. The process of claim 1, wherein the incubation period is about 1 minute to about 10 minutes.

7. A process for producing cDNA from isolated RNA, comprising:
providing isolated RNA from which cDNA is desired to be produced;
contacting the RNA with a suitable buffer, a suitable amount of deoxynucleotides and a reverse transcriptase from Simian Immunodeficiency Virus-agm.sab to form a mixture;
incubating the mixture at an incubation temperature of about 45° C. to about 65° C. for an incubation period of between about 1 minute to about 20 minutes; wherein the RNA is reverse transcribed to cDNA; and
contacting the cDNA with a suitable buffer, a suitable amount of deoxynucleotides and a DNA polymerase capable of amplifying the cDNA under conditions suitable for polymerase chain reaction; and
amplifying the cDNA through a suitable polymerase chain reaction protocol.

8. The process of claim 7, wherein the reverse transcriptase has an amino acid sequence comprising an amino acid sequence having at least about 90% sequence similarity to SEQ ID NO:2.

9. The process of claim 7, wherein the reverse transcriptase has an amino acid sequence comprising an amino acid sequence having at least about 95% sequence similarity to SEQ ID NO:2.

10. The process of claim 7, wherein the reverse transcriptase has an amino acid sequence comprising SEQ ID NO.2

11. The process of claim 7, wherein the incubation temperature is about 55° C. to about 60° C.

12. The process of claim 7, wherein the incubation period is about 1 minute to about 10 minutes.

13. A process for producing cDNA from isolated RNA, comprising:
providing isolated RNA from which cDNA is desired to be produced;
contacting the RNA with a buffer suitable for the performance of both reverse transcription and polymerase chain reaction, a suitable amount of deoxynucleotides a reverse transcriptase from Simian Immunodeficiency Virus-agm.sab to form a mixture and a suitable DNA polymerase capable of amplifying cDNA under conditions suitable for polymerase chain reaction;
incubating the mixture at an incubation temperature of about 45° C. to about 65° C. for an incubation period of between about 1 minute to about 20 minutes; wherein the RNA is reverse transcribed to cDNA; and
amplifying the cDNA through a suitable polymerase chain reaction protocol.

14. The process of claim 13, wherein the reverse transcriptase has an amino acid sequence comprising an amino acid sequence having at least about 90% sequence similarity to SEQ ID NO.2.

15. The process of claim 13, wherein the reverse transcriptase has an amino acid sequence comprising an amino acid sequence having at least about 95% sequence similarity to SEQ ID NO:2.

16. The process of claim 13, wherein the reverse transcriptase has an amino acid sequence comprising SEQ ID NO.2

17. The process of claim 13, wherein the incubation temperature is about 55° C. to about 60° C.

18. The process of claim 13, wherein the incubation period is about 1 minute to about 10 minutes.

19. A kit for producing cDNA from isolated RNA, comprising:
a buffer suitable for the performance of both reverse transcription and polymerase chain reaction;
a suitable amount of deoxynucleotides;
a reverse transcriptase from Simian Immunodeficiency Virus-agm.sab to form a mixture;
and a suitable DNA polymerase capable of amplifying cDNA under conditions required for polymerase chain reaction.


SEQUENCE LISTING (2 sequences)

SEQ ID NO: 1 - DNA, 1680 bp, Simian Immunodeficiency Virus
acacaaagagagatagaacctataaaagtccacttaaagccaggacaagatgggccaaggataaggcaatggcctttgtcaaaagaaaaaatagaggccttaaaggccatttgtgaagacttagaaaaacaaggacatttggaaagaatagggccagaaaatccatataacaccccagtctttgcaataaggaagaaagataaaactcagtggagaatactcatggacttcaggcagttaaataaaagtactcaagactttcaggaagtccagttagggataccccacccagcaggcttacagcaaagggagcagattacagtgttggacataggagatgcctattttagctgccctttagatccagactttcagaaatatacagcattcaccatcccatcagtcaataatagggagccaggcataagatatcagtataaggtgctcccacaggggtggaaaggctcacccacaatttttcagacaacagccaacaaaattctgcaggaatttaggcaaaagaacccagatgtagatatatatcaatacatggatgatatgttaatagccagtgataggccaaaggcagaacatctagtaatggtacagcagttaagagactatctagaaacttgggggtttaaaacccctgaaaagaagtttcaaaaggatccaccatacctctggatggggtatgaattgtatccaaagaaatggcaactgcaggagatcaccctaccagaaagggaagaatggacagtgaatgatattcaaaaattagtaggaaagttaaattgggcaagtcaaatttacacagggataaagactaaacatttgtgtagactaattagaggagctagacctttgacagagatagtccaatggacagaagaggcagaactagaattagaagaaaatagacagatcttaagacaaaagcagcagggacaatattatgaccctgctcttcctttaagggctaaggttcttaaactgggagatggccaatggggatatcagatataccagccagaaaataaaatactgaaggttggcaaatatgcaaaaatcaaaacagcacacaccaatgaattaagaatgttagcaggcctagtacagaagataggaaaggaaagcatagtcatctggggacagatacctattatggaactgccagtagaaagagagctctgggaacaatggtggtctgactattggcaggtcacctggatcccggaatgggaaatggtcagtactcctcaattgattagattgtggtacaaattagtaaaagatcccatcccaggagaagcagtctattatgtagatggggcagctaatagaaattccaaagaaggaaaagcaggatacttaacagatagaggggatcaaaaggtggtagcattagagaatactaccaaccagaaagcagagttagaggccattctgttagccttaagagactctggaagcaaagtaaatataataacagactcacaatatgctatgggtattatagcaggggaacctacagaatcagataataacatagtacaacaaattatagaggaactaataaagaaggaggcagtgtatatagcatgggtacctgcccataaaggagtaggtggcaatgaggaaattgataaactagttagtcaaggaataagacaagtacta

SEQ ID NO: 2 - protein, 560 aa, Simian Immunodeficiency Virus
TQREIEPIKVHLKPGQDGPRIRQWPLSKEKIEALKAICEDLEKQGHLERIGPENPYNTPVFAIRKKDKTQWRILMDFRQLNKSTQDFQEVQLGIPHPAGLQQREQITVLDIGDAYFSCPLDPDFQKYTAFTIPSVNNREPGIRYQYKVLPQGWKGSPTIFQTTANKILQEFRQKNPDVDIYQYMDDMLIASDRPKAEHLVMVQQLRDYLETWGFKTPEKKFQKDPPYLWMGYELYPKKWQLQEITLPEREEWTVNDIQKLVGKLNWASQIYTGIKTKHLCRLIRGARPLTEIVQWTEEAELELEENRQILRQKQQGQYYDPALPLRAKVLKLGDGQWGYQIYQPENKILKVGKYAKIKTAHTNELRMLAGLVQKIGKESIVIWGQIPIMELPVERELWEQWWSDYWQVTWIPEWEMVSTPQLIRLWYKLVKDPIPGEAVYYVDGAANRNSKEGKAGYLTDRGDQKVVALENTTNQKAELEAILLALRDSGSKVNIITDSQYAMGIIAGEPTESDNNIVQQIIEELIKKEAVYIAWVPAHKGVGGNEEIDKLVSQGIRQVL